(19) United States
(12) Patent Application Publication
(10) Pub. No.: US 2025/0210180 A1
(43) Pub. Date: Jun. 26, 2025

(54) METHOD AND SYSTEM FOR PREDICTING AND/OR DETECTING THE PRESENCE OF A LESION

(71) Applicant: Hoffmann-La Roche Inc., Little Falls, NJ (US)

(72) Inventors: Thomas Felix ALBRECHT, Riehen (CH); Josep ARUS POUS, Sabadell (ES); Yaniv COHEN, Riehen (CH); Juergen Michael FUNK, Lörrach (DE); Vanessa Lea SCHUMACHER, Efringen-Kirchen (DE); Grégoire CLÉMENT, Zurich (CH)

(73) Assignee: Hoffmann-La Roche Inc., Little Falls, NJ (US)

(21) Appl. No.: 19/075,267

(22) Filed: Mar. 10, 2025

Related U.S. Application Data
(63) Continuation of application No. PCT/EP2023/084749, filed on Dec. 7, 2023.

(30) Foreign Application Priority Data
Dec. 8, 2022 (EP) 22212250.9

(51) Int. Cl. G16H 30/20; G06N 3/0464; G16H 50/20

(52) U.S. Cl. CPC G16H 30/20; G06N 3/0464; G16H 50/20

(57) ABSTRACT

The present disclosure relates to a computer-implemented method of predicting and/or detecting the presence of a lesion in a human or animal tissue, the method comprising: identifying a tissue type based on image data of the human or animal tissue using a first model, and predicting and/or detecting the presence of a lesion in the tissue based on the image data and the tissue type using a second model.

### CROSS-REFERENCE TO RELATED APPLICATIONS

This application is a continuation application under 35 U.S.C. § 120 of International Patent Application No. PCT/EP2023/084749, filed Dec. 7, 2023, which claims priority under 35 U.S.C. § 119 (a) and/or PCT Article 8 to European Patent Application No. 22212250.9, filed Dec. 8, 2022. The entire disclosures of International Patent Application No. PCT/EP2023/084749 and European Patent Application No. 22212250.9 are incorporated herein by reference.

### TECHNICAL FIELD

The present application generally relates to the field of computational pathology and in particular to the prediction of the presence of a lesion in a human or animal tissue using artificial intelligence (AI).

### BACKGROUND

Artificial Intelligence (AI) systems have the potential to empower pathologists by automating parts of toxicology studies, resulting in considerable time gains during pre-clinical assessment of new compounds. Machine learning (ML) offers the possibility to train such systems without the need for costly pixel-wise annotations.

Computational pathology describes an approach to diagnosis incorporating multiple sources of digital data. A key element of the approach is the ability to derive data from histopathology images as for example whole-slide imaging (WSI) of stained tissue sections. It has been shown (Holger Hoefling et al., “HistoNet: A Deep Learning—Based Model of Normal Histology”, Toxicologic Pathology 2021, Vol. 49 (4) 784-797) that a comprehensive set of tissues can be recognized by standard convolutional neural networks (CNNs) trained on small images or patches extracted at various magnifications from H & E-stained WSI of a diversity of rat tissues.

It is desirable to predict the presence of lesions in tissues from digital pathology images, in particular WSI images, that could easily be interpreted by pathologists, and deliver the solution at ease and scale through automation.

The present disclosure is directed to provide improved methods for lesion detection and prediction in digital histological images of human or animal tissue.

### SUMMARY

A simplified summary of some embodiments of the disclosure are provided in the following to give a basic understanding of these embodiments and their advantages. Further embodiments and technical details are described in the detailed description presented below.

According to an embodiment, a computer-implemented method of predicting and/or detecting the presence of a lesion in a human or animal tissue, the method comprising: identifying a tissue type based on image data of the human or animal tissue using a first model, and predicting and/or detecting the presence of a lesion in the tissue based on the image data and the tissue type using a second model. This method allows an improved prediction of the presence of a lesion in a human or animal tissue. In particular, since the tissue type is automatically taken into account, the reliability of the prediction can be increased, as the second model can be trained for lesion detection in a particular tissue type.

The method may thus be referred to as a detection method, as it may serve to (e.g., automatically) detect the presence of a lesion. For example, the detection result may be the output of a system according to the present disclosure. However, the method may also be referred to as a prediction method, as the method (e.g., trained machine learning) models and may thus only provide a prediction (or estimation) of the factual characteristics of the tissue.

The image data may be in particular histological image data.

For example, the first model may perform a classification of the tissue type, e.g., determine a probability value that the inputted test data set corresponds to a specific tissue type.

The second model may perform a binary classification, i.e., distinguish between “lesion detected” and “no lesion detected”.

Identifying a tissue type may comprise inputting the image data into the first model, wherein in response the first model identifies a tissue type.

Predicting and/or detecting the presence of a lesion may comprise predicting and/or detecting at least one of: a probability of the presence of a lesion, a lesion type and/or a degree of lesion severity.

Predicting and/or detecting the presence of a lesion may comprise predicting and/or detecting for each of a plurality of different tissue regions a probability of the presence of a lesion.

For example, predicting and/or detecting the presence of a lesion may comprise generating a first (e.g., positive) heatmap of the tissue, wherein the heatmap indicates for each of a plurality of different tissue regions a probability of the presence of a lesion.

Furthermore, predicting and/or detecting the presence of a lesion may comprise generating a second (e.g., negative) heatmap of the tissue, wherein the second heatmap indicates for each of a plurality of different tissue regions a probability of the presence of no lesion.

Predicting and/or detecting the presence of a lesion may comprise generating a superimposed heatmap of the tissue combining the first heatmap with the second heatmap by superimposition.

At least one of the first, second and superimposed heatmaps may be related to the probability of the presence of a particular lesion type.

Predicting and/or detecting the presence of a lesion may comprise generating a plurality of heatmaps, wherein each heatmap is related to the probability of the presence of a particular lesion type.

At least one of the first, second, superimposed, and the plurality of heatmap may highlight those regions which are most critical for a prediction of whether the tissue contains a lesion or not.

Accordingly, pathologists may faster determine whether the tissue comprises a lesion or not, by being guided to decision-relevant regions in the tissue. For example, the second model may have learnt during training to highlight such regions in a specific tissue type which most frequently contain lesions, even though in the particular case no lesion has been predicted. This effect is in particular possible, in case the second model is a transformer-based model and/or an attention-based model, which does not process single regions in an isolated manner, but takes adjacent regions into account.

At least one of the first and second models may be a machine learning (ML) model, in particular a deep learning (DL) model.

The first model may be or may comprise a convolutional neural network.

The second model may comprise a transformer-based model and optionally a convolutional neural network for preprocessing the input of the transformer-based model. The transformer-based model may also be replaced by an attention-based model, i.e. a machine-learning model comprising an attention mechanism, e.g. an attention-based multiple instance learning (MIL) model, as described in M. Ilse et. al. Attention-based Deep Multiple Instance Learning, 2018, arXiv: 1802.04712. The convolutional neural network (CNN) may in particular reduce the size of the input data, by processing the original image data (i.e., its input data) to smaller embeddings (being the output of the CNN). In other words, the CNN may transform the input data into a shape that is smaller in size but ideally contains all of the relevant information.

At least one of the first, second, superimposed, and the plurality of heatmaps is generated using the transformer-based model and/or the attention-based model.

Accordingly, the predictions become more reliable, as the second model does not process single regions in an isolated manner, but takes adjacent regions (i.e., the context) into account.

The convolutional neural network may be trained to identify a particular tissue type in a plurality of training data sets of digital histological images of human or animal tissue.

Optionally, the method may further comprise: inputting a test data set of digital histological images of human or animal tissue into the trained convolutional neural network, receiving as an output result of the convolutional neural network a probability value that the inputted test data set corresponds to the target tissue type.

The training of the convolutional neural network may comprise performing with the plurality of training data sets of digital histological images of human or animal tissue the steps of selecting a target tissue area of a training data set, dividing the target tissue area into a first set of tiles of constant size and having a first image magnification, dividing the target tissue area into at least a second set of tiles of constant size and having a second image magnification different from the first image magnification, inputting the at least two sets of tiles into the convolutional neural network, wherein the convolutional neural network is an at least two-headed convolutional neural network in which the at least two sets of tiles are processed in parallel and whereby the features of the at least two sets of tiles are concatenated, and labelling the output results of the convolutional neural network with respect to the target tissue type. This method allows an improved identification of different tissue types.

In some embodiments the bit size of all sets of tiles are identical, for example 224×224×3 pixels.

In some embodiments the centroids of the different sets of tiles are identical.

In some embodiments the training data sets and test data sets of digital histological images of human or animal tissue are whole slide images (WSI).

In some embodiments the identified different tissue types are tissues of different organs.

In some embodiments dividing the target tissue area into the extraction of the at least two tile sets comprise extracting a foreground mask of the tissue region, providing annotations classifying areas of the tissue region, and merging the annotations with the foreground mask. This procedure provides a reliable method of dividing the target tissue area into standardized tiles.

In some embodiments the at least two different sets of tiles correspond to image magnification factors of 1.25, 5, and 10.

Some embodiments comprise applying a binary training model for identification of a particular tissue type or organ.

In some embodiments the training procedure of the convolutional neural network comprises random horizontal and/or vertical flips of the tiles.

In some embodiments the training procedure of the convolutional neural network comprises variations of the color, hue, saturation, brightness and/or contrast of the tile images.

The present disclosure may further relate to a computer program comprising computer-readable instructions which when executed by a data processing system cause the data processing system to carry out the method of the present disclosure.

The present disclosure may further relate to a recording medium readable by a computer and having recorded thereon a computer program including instructions for executing the steps of a method of the present disclosure.

The present disclosure further relates to a system for predicting and/or detecting the presence of a lesion in a human or animal tissue, the system comprising a processing unit configured to:
input image data of human or animal tissue into a first model, wherein in response the first model identifies a tissue type, and
predict the presence of a lesion based on the image data and the tissue type using a second model.

### BRIEF DESCRIPTION OF THE DRAWINGS

The foregoing summary as well as the following detailed description of preferred embodiments are better understood when read in conjunction with the append drawings. For illustrating the embodiments, the drawings show exemplary details of systems, methods, and experimental data. The information shown in the drawings is exemplary and explanatory only and is not restrictive of the invention as claimed. In the drawings:

FIG. 1 shows a flow chart illustrating an embodiment of a computer-implemented method of predicting and/or detecting the presence of a lesion in a human or animal tissue;

FIG. 2 is a schematic illustration of an embodiment of a model for use with prediction method of FIG. 1;

FIG. 3 shows a flow chart illustrating an embodiment of a computer-implemented method of identifying a tissue type in data sets of digital histological images;

FIG. 4 shows a flow chart illustrating an embodiment of a method of training a convolutional neural network of the identification method of FIG. 3;

FIG. 5 shows exemplary images of different tissue types at different magnifications;

FIG. 6 is a schematic illustration of an embodiment of a convolutional neural network for use with identification method of FIG. 3;

FIG. 7 is a schematic illustration of exemplary tiles showing tissue areas at different magnifications;

FIG. 8 is a schematic illustration of dividing a WSI image into different sets of tiles;

FIG. 9 shows a flow chart illustrating an embodiment of a method of dividing a target tissue area into a plurality of tile sets of the identification method of FIG. 3;

FIG. 10 illustrates a training and a test procedure in an embodiment of the identification method of FIG. 3; and

FIG. 11 is a schematic block diagram illustrating applications of the identification method according to FIGS. 3-10.

### DETAILED DESCRIPTION

The reliable automated prediction and/or detection of the presence of a lesion in a human or animal tissue from digital pathology images, in particular WSI images, is highly desirable for different preclinical working environments, in particular for a faster and anyhow reliable and precise diagnosis by pathologists. The scope of the present disclosure is thus multifold, addressing in particular the following three main aspects: optimization of model performance of the lesion detection, generation of predictions that could easily be interpreted by pathologists, and delivery of the solution at ease and scale through automation.

The present disclosure therefore further proposes to predict the presence of a lesion in a human or animal tissue, the method comprising: identifying a tissue type based on image data of the human or animal tissue using a first model, and predicting and/or detecting the presence of a lesion in the tissue based on the image data and the tissue type using a second model. This method allows an improved prediction of the presence of a lesion in a human or animal tissue. In particular, since the tissue type is automatically taken into account, the reliability of the prediction can be increased, as the second model can be trained for lesion detection in a particular tissue type.

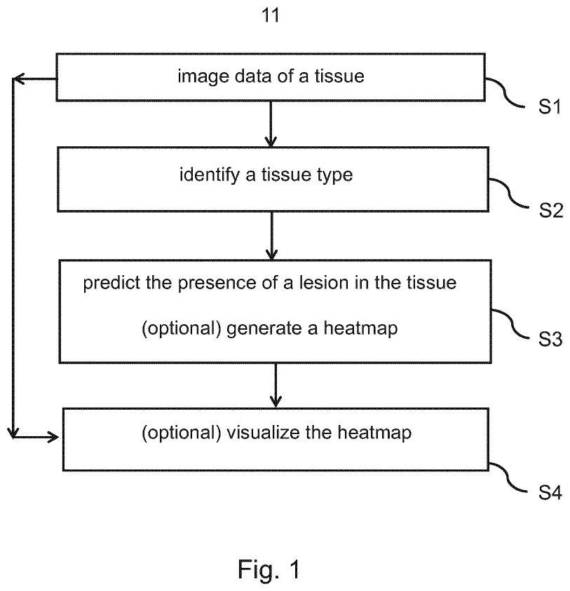

FIG. 1 shows a flow chart illustrating an embodiment of a computer-implemented method of predicting and/or detecting the presence of a lesion in a human or animal tissue.

In an operation S1 image data of a tissue are provided. The image data may for example be in particular histological image data of a human or an animal. The data may be provided for example by a data storage, a data interface, an image sensor, or other.

The image data may be for example whole-slide images (WSI). The WSI may be preprocessed in operation S1, such that tiles of the WSI are extracted.

The outputs are a prediction of the different lesion types present in the WSI and a heatmap for explainability. The latter can be visualized, for example, on the PMA Studio visualization tool.

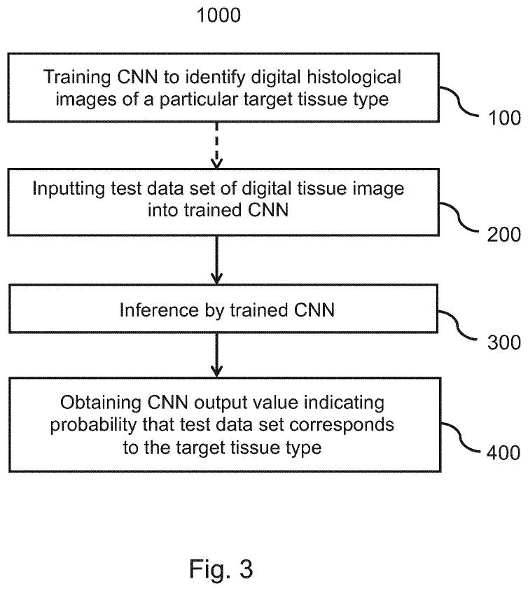

In an operation S2 a tissue type is identified based on the image data using a first model. Exemplary methods of identifying a tissue type (e.g., an organ type) are described in context of FIGS. 3 to 11 below. For example, the first model may perform a classification of the tissue type, e.g., determine a probability value that the inputted test data set corresponds to a specific tissue type. An exemplary use case of identifying an organ type is known from Citlalli Gámez Serna et. al., MMO-Net (Multi-Magnification Organ Network): A use case for Organ Identification using Multiple Magnifications in Preclinical Pathology Studies, Journal of Pathology Informatics, Volume 13, 2022, 100126, ISSN 2153-3539.

In an operation S3 the presence of a lesion is predicted in the tissue based on the image data and the tissue type using a second model. Accordingly, the first model may provide the tissue type to the second model. The second model may be trained to predict lesions in exactly this tissue type. For example, the second model may comprise one or several sub-models, which are each trained on image data of different tissue types. In this case the best matching sub-model may be selected based on the identified tissue type. Accordingly, since the second model may be trained for lesion detection in the identified tissue type, the reliability of the prediction can be increased.

For example, the tiles (extracted in operation S1) of a targeted tissue type (e.g., of a particular organ identified in operation S2) may be used as inputs for the lesion detection transformer.

Optionally, operation S3 may further comprise generating a heatmap of the tissue.

Accordingly, the outputs of the second model may be a prediction of the different lesion types present in the WSI and/or a heatmap for explainability.

For example, the heatmap may be or comprise a (first) positive heatmap, which indicates for each of a plurality of different tissue regions a probability of the presence of a lesion. The second model may thus be trained on positive examples, i.e. images of sample tissues having somewhere a lesion.

Furthermore, for example, the heatmap may be or comprise a (second) negative heatmap of the tissue, which indicates for each of a plurality of different tissue regions a probability of the presence of no lesion. The second model may thus be trained (in addition) on negative examples, i.e. images of sampled tissues having no lesion. The negative heatmap may for example highlight those regions which are most critical for a prediction that the tissue contains no lesion. These regions may be for example those which in the training data of sample tissue most frequently contained a lesion.

A superimposed heatmap of the tissue may be obtained by combining the first heatmap with the second heatmap by superimposition. The superimposed heatmap may also be obtained by a second model which is trained on both positive and negative examples. It is also possible that a plurality of heatmaps are generated, wherein each heatmap is related to the probability of the presence of a particular lesion type.

Accordingly, pathologists may faster determine whether the tissue comprises a lesion or not, by being guided to decision-relevant regions in the tissue. For example, the second model may have learnt during training to highlight such regions in a specific tissue type which most frequently contain lesions, even though in the particular case no lesion has been predicted. This effect is in particular possible, in case the second model is a transformer-based model and/or an attention-based model, which does not process single regions in an isolated manner, but takes adjacent regions into account. In an optional step S4 the heatmap may be visualized. For example, the heatmap may be visualized on a PMA Studio visualization tool. The heatmap may for example be displayed beside the image of the tissue (e.g., an WSI) or in a superimposed manner.

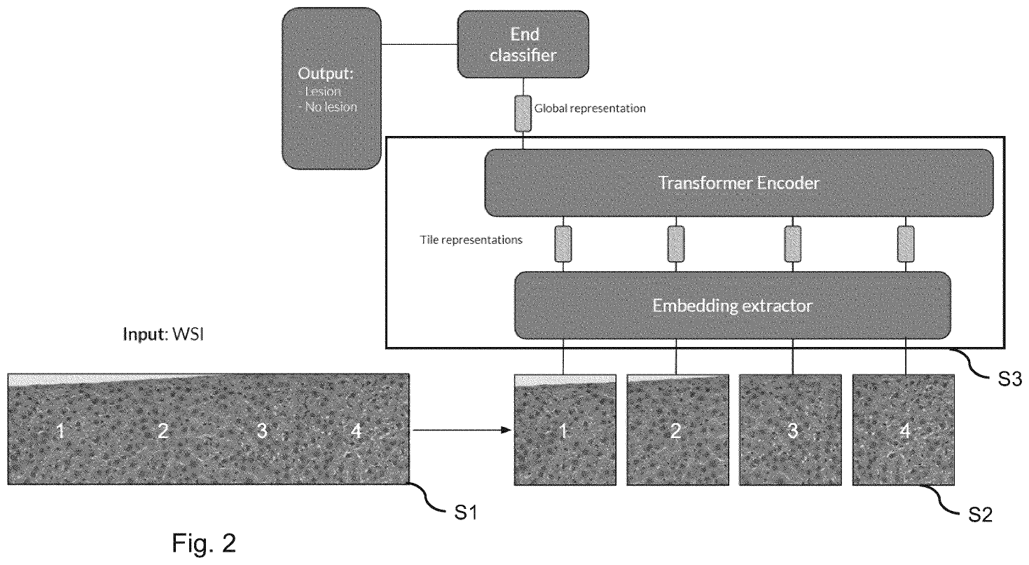

FIG. 2 is a schematic illustration of an embodiment of a (first and second) model architecture for use with the prediction method of FIG. 1.

Due to the sheer size of whole-slide images (WSI), it may be desirable to decompose the images in smaller elements, i.e. tiles, and process them individually before aggregating them for a downstream task performed by the first and/or second model (cf. S1 and S2). The single tiles may be annotated with an identified tissue type (cf. S2).

The tiles may then be processed by a second model comprising for example a transformer encoder model and optionally an embedding extractor model (cf. S3).

Accordingly, a transformer architecture may be leveraged in the second model. The Transformer is highly performing deep learning architecture introduced in 2017 by Vaswani et al., cf.: Vaswani, A., Shazeer, N., Parmar, N., Uszkoreit, J., Jones, L., Gomez, A. N., Kaiser, Ł. and Polosukhin, I., 2017. Attention is all you need. In Advances in neural information processing systems (pp. 5998-6008). The transformer architecture was initially designed for Natural Processing Tasks (NLP) such as language-to-language translation. The application of transformer models on images is described in e.g.: Dosovitskiy, A., Beyer, L., Kolesnikov, A., Weissenborn, D., Zhai, X., Unterthiner, T., Dehghani, M., Minderer, M., Heigold, G., Gelly, S. and Uszkoreit, J., 2020 September. An Image is Worth 16×16 Words: Transformers for Image Recognition at Scale. In International Conference on Learning Representations.

However, it may be desirable to use an optional embedding extractor model for obtaining compressed tile representations (embeddings) as input for the second model instead of the full tile. These embeddings may be obtained by running an embedding extractor model in the form of a Convolution Neural Network on all the WSI tiles and saving them for further reuse. Using for example a Resnet50 architecture pre-trained on ImageNet with e.g., the BYOL training strategy, a tile feature vector may be reduced to 2048 dimensions, being almost 100× smaller than the initial version. These parameters may be further compressed by using linear projection layers in the model architecture.

Even though in this approach the embeddings may be “frozen”, i.e., fixed, the Transformer layers may be improved in a learning process. Moreover, the approach allows for augmenting the data at the tiles level and reaches good predictive abilities.

The second model may output based on the plurality of tiles of a WSI (or the respective tile representations) a global representation. Said global representation may be fed into an end classifier (for example a binary classifier) which predicts the presence of a lesion in the tissue of the WSI.

Unlike traditional multiple instance learning approaches, transformers may not offer a straightforward way to interpret their predictions at the tile level. It is therefore proposed to adapt the second model to extract the relative importance it gives to each tile by evaluating the attention values reported for each embedding. The tiles/attention values may thus be aggregated across the multiple attention layers. An optimized global representation may be learnt by the second model with the help of human annotators (e.g., pathologists) during training, in order to output meaningful heatmaps.

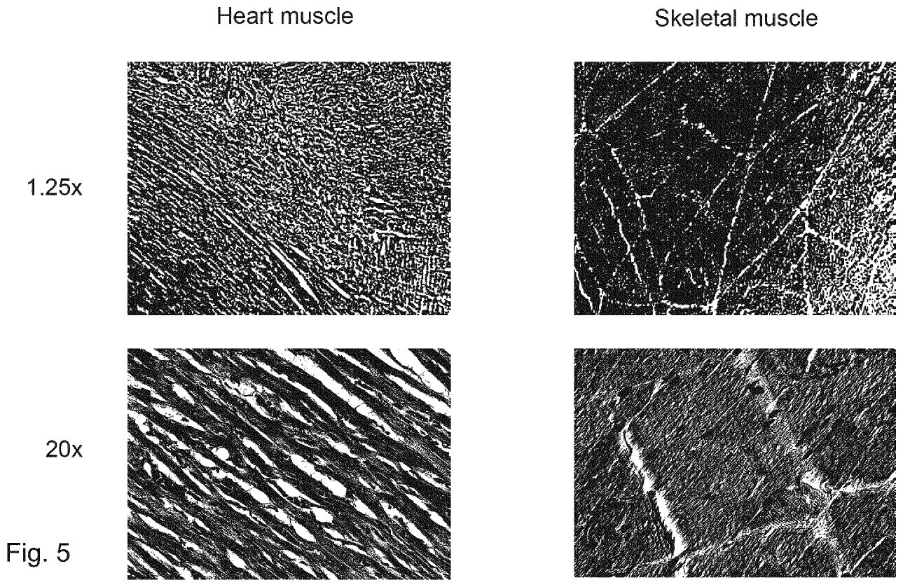

The reliable automated identification of different tissue types and in particular the identification of organs in pathological images is highly desirable for different preclinical working environments, in particular for a reliable prediction of the presence of a lesion in a human or animal tissue. This identification of different organs or tissue types depends on the magnification of the digital histologic images as for example the WSI images. While some organs show characteristic structures at low image magnifications of e.g., 1.25×, other organs can be best identified at higher magnifications such as 5× or 10×. This is illustrated in FIG. 5, which shows an example of whole slides images (WSI) of two different tissue types (heart muscle and skeletal muscle tissue) at two different magnifications, namely 1.25× and 20×. While the tissue structure of both muscle tissue types looks very similar at the lower magnification of 1.25×, the structural differences are more prominent at the larger magnification of 20×. Consequently, an automated tissue identification using a machine learning model will obtain better results distinguishing heart muscle tissue from skeletal muscle tissue at the higher image magnification level than at the lower magnification level. This relation, however, cannot be generalized. For some tissue types the identification is easier at lower magnification, e.g., because more “context” is visible in the images of lower magnification.

The present disclosure therefore further proposes to train a convolutional neural network (CNN) for tissue type identification using different image magnifications in parallel. In particular, a computer-implemented method of identifying a tissue type in data sets of digital histological images using a training procedure of a convolutional neural network comprises performing with a plurality of training data sets the steps of selecting a target tissue area of the training data set, dividing the target tissue area into a different sets of tiles of constant size but having different image magnifications, and inputting the sets of tiles into a multi-headed convolutional neural network, wherein the sets of tiles having different image magnifications are processed in parallel and the features of the sets of tiles are concatenated. With this training procedure the tissue type or organ identification accuracy can be improved since tissue features more characteristic at lower magnifications as well as those more characteristic at higher magnifications contribute to the learning procedure of the convolutional neural network. Preferably, the selection of the number of different tile sets and their respective image magnifications can be adapted and optimized to the respective target tissue or target organ.

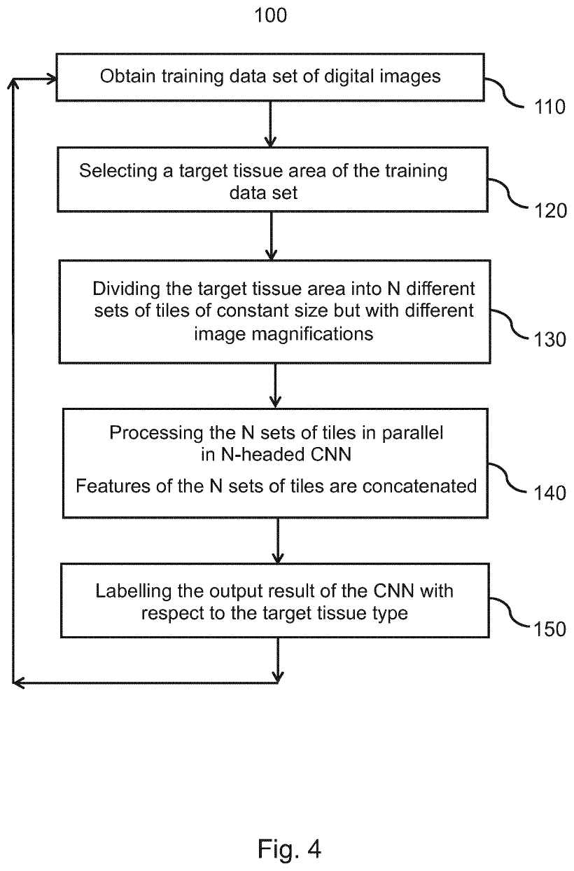

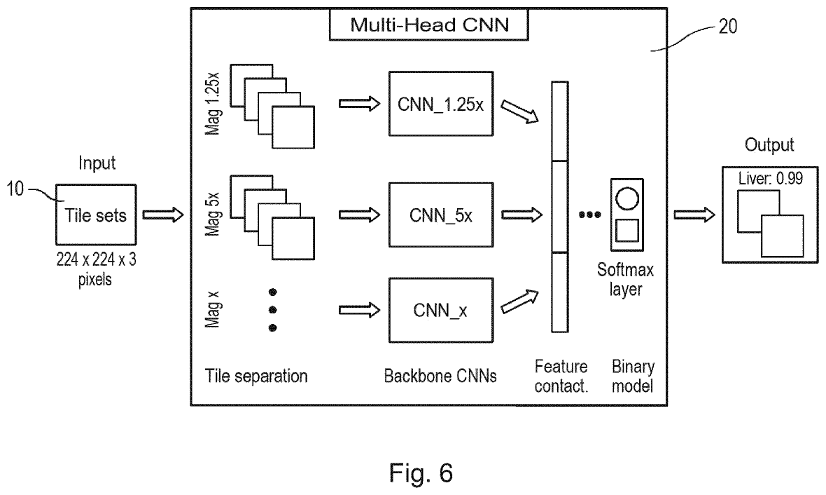

FIG. 3 is a flow chart illustrating an embodiment of a computer-implemented method 1000 of identifying a tissue type in data sets of digital histological images, for example WSI images. These are available in large numbers in archives for example for rat samples. In a first step 100 a convolutional neural network CNN is trained to identify images of a particular target tissue type, e.g. a particular organ as for example liver, kidney, or salivary gland. The procedure of training the neural network is in more detail explained later with reference to FIGS. 4 and 6. In a second step 200 of the identification method a test data set also preferably consisting of WSI images is input into the trained CNN, which performs the inference in subsequent method step 300. In step 400 a result is obtained which provides a probability that the input test data set shows tissue of the target tissue type, for example liver tissue.

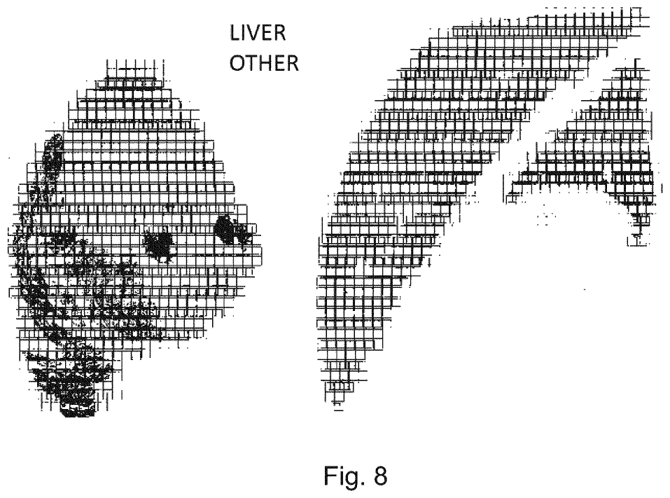

FIG. 4 is a flow chart illustrating an embodiment of a method of training the CNN referred to in FIG. 3. In a first step 110 a training data set of digital images, preferably WSI images, is obtained. In a second step 120 a target tissue area is selected for the training procedure by means of manual annotations. In the subsequent method step 130 the target tissue area is divided into different sets of tiles as illustrated in FIG. 8. Each tile preferably has a constant pixel size of e.g., 224×224×3 pixels. Standardization of the tile size and format can improve the accuracy of organ detection. The selected target tissue area is divided into at least two different sets of tiles of constant pixel size but of different image magnification. The target area is thus for example divided into a first set of tiles (of e.g. 224×224×3 pixels) having an image magnification of 1.25, a second set of tiles (of also 224×224×3 pixels) having an image magnification of 5, and a third set of tiles (of 224×224×3 pixels) having an image magnification of 10. The number of tile sets and the magnification levels can be selected depending on the specific target tissue or organ to which the CNN is to be trained for.

In the next step 140 (FIG. 4) of the training procedure the N (typically 2 or 3) sets of tiles are submitted to the input of an N-headed CNN. Thus, the data sets based on different image magnifications are processed in parallel and the features of the N sets of tiles are concatenated. An embodiment of a corresponding CNN is schematically illustrated in FIG. 6. The tile sets 10 are input into the multi-head CNN 20, which processes the tile sets of different magnification in parallel using separate backbone CNNs. The features are concatenated and an output result is delivered at an output. If a binary model is used, a binary labelling is performed during the training procedure, i.e., labeling “yes” or “no” with respect to a target organ like a liver (step 150 in FIG. 4).

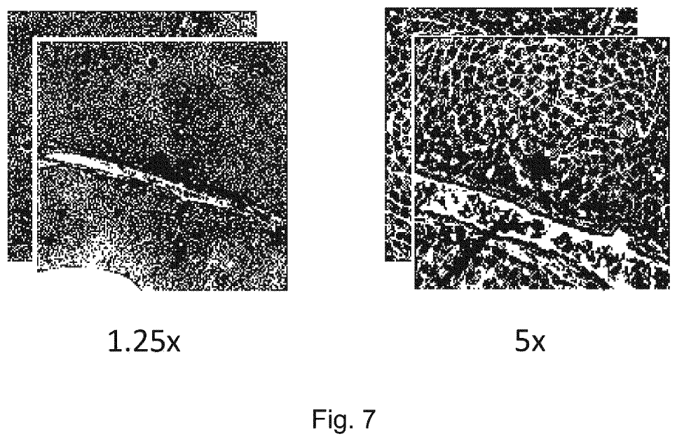

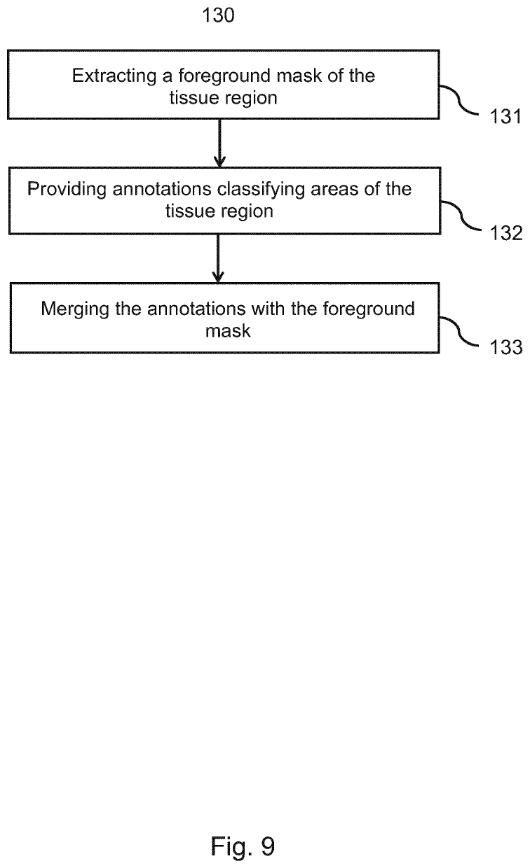

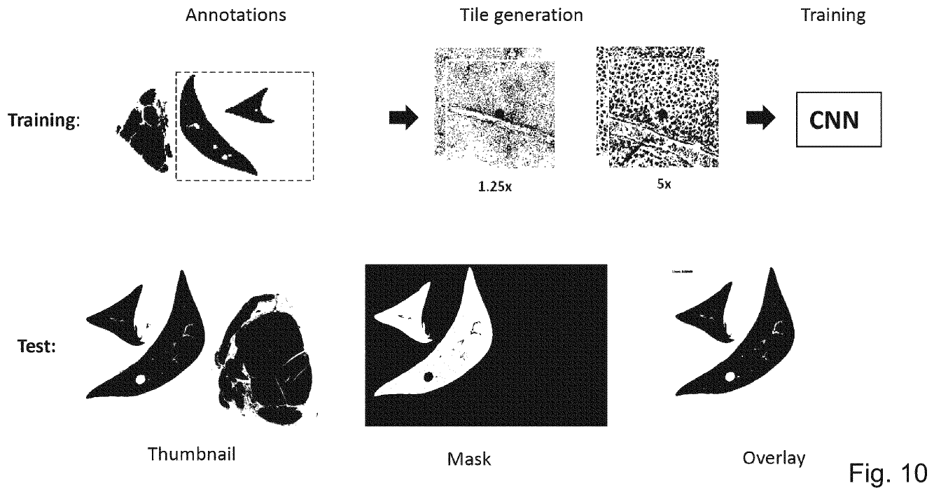

The method step 130 (FIG. 4) of dividing a target tissue area into sets of tiles is now explained in connection with FIGS. 7 to 10. In a first step 131 (FIG. 9) a foreground mask of the tissue region (liver) is extracted as illustrated in FIG. 10. In step 132 annotations classifying areas of the tissue region are provided and an overlay or merge of the foreground mask with the annotations is performed (step 133 in FIG. 9 and bottom-right image in FIG. 10). The obtained tile sets are illustrated in FIG. 8. These comprise tiles showing only part of the target tissue type as the liver and tiles covering boundary regions containing both liver tissue and non-liver tissue. Preferably the different tile sets representing different image magnifications have the same centroid as illustrated in FIG. 7 showing the centroid as a red dot.

In order to improve the robustness of the organ detection, different augmentation techniques can be applied for the training procedure including random horizontal or vertical tile flipping, random color augmentation and/or variation of hue, saturation, brightness, and contrast of the tile image.

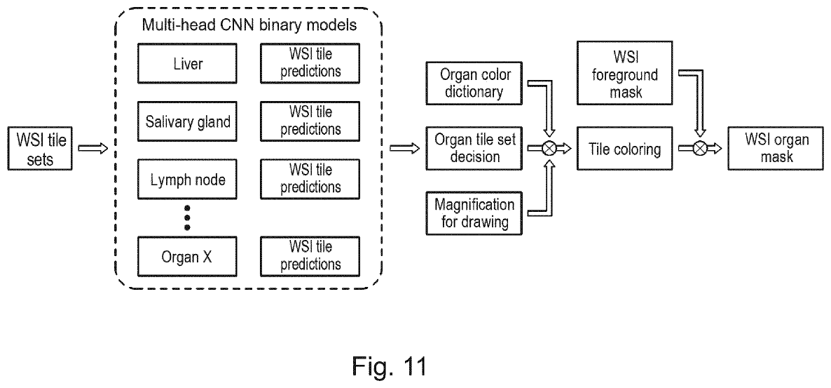

FIG. 11 schematically illustrates a procedure of obtaining WSI organ masks. Based on WSI tile sets, binary identification models of different types of organs and tissue types can be obtained by training multi-headed CNNs. With the obtained tile set decision (identification) and an organ color dictionary and selecting a drawing magnification a tile coloring can be obtained, which in turn can be combined with a WSI foreground mask to obtain a WSI organ mask.

Applications of the identification methods are numerous. Based on WSI image tile sets, binary identification models of different types of organs and tissue types can be obtained by training multi-headed CNNs. These include the liver, salivary gland, lymph nodes, kidney, urinary bladder, etc. but also for example different muscle types or models directed to distinguish between thyroid and parathyroid glands.

Aspects of this disclosure including the CNN can be implemented in digital circuits, computer-readable storage media, as one or more computer programs, or a combination of one or more of the foregoing. The computer-readable storage media can be non-transitory, e.g., as one or more instructions executable by a cloud computing platform and stored on a tangible storage device.

Unless otherwise stated, the foregoing alternative examples are not mutually exclusive, but may be implemented in various combinations to achieve unique advantages. In the foregoing description, the provision of the examples described, as well as clauses phrased as “such as,” “including” and the like, should not be interpreted as limiting embodiments to the specific examples; rather, the examples are intended to illustrate only one of many possible embodiments.

Further implementations are summarized in the following examples.

Example 1: A computer-implemented method of predicting and/or detecting the presence of a lesion in a human or animal tissue, the method comprising: identifying a tissue type based on image data of the human or animal tissue using a first model, and predicting and/or detecting the presence of a lesion in the tissue based on the image data and the tissue type using a second model.

Example 2: The method of example 1, wherein identifying a tissue type comprises inputting the image data into the first model, wherein in response the first model identifies a tissue type, and/or predicting and/or detecting the presence of a lesion comprises inputting the image data into the second model, the image data being annotated with the tissue type and/or comprise data representing the tissue type, wherein in response the second model predicts the presence of a lesion in the tissue.

Example 3: The method of example 1 or 2, wherein predicting and/or detecting the presence of a lesion comprises predicting and/or detecting at least one of: a probability of the presence of a lesion, a lesion type and/or a degree of lesion severity.

Example 4: The method of one of the preceding examples, wherein predicting and/or detecting the presence of a lesion comprises predicting and/or detecting for each of a plurality of different tissue regions a probability of the presence of a lesion, and/or predicting and/or detecting the presence of a lesion comprises generating a first heat heatmap of the tissue, wherein the heat heatmap indicates for each of a plurality of different tissue regions a probability of the presence of a lesion.

Example 5: The method of one of the preceding examples, wherein predicting and/or detecting the presence of a lesion comprises generating a second heat heatmap of the tissue, wherein the second heat heatmap indicates for each of a plurality of different tissue regions a probability of the presence of no lesion.

Example 6: The method of one of the preceding examples, wherein predicting and/or detecting the presence of a lesion further comprises generating a superimposed heat heatmap of the tissue combining the first heat heatmap with the second heat heatmap by superimposition.

Example 7: The method of one of the preceding examples, wherein at least one of the first, second and superimposed heat heatmap is related to the probability of the presence of a particular lesion type, and/or predicting and/or detecting the presence of a lesion further comprises generating a plurality of heat heatmaps, wherein each heat heatmap is related to the probability of the presence of a particular lesion type.

Example 8: The method of one of the preceding examples, wherein at least one of the first, second, superimposed, and the plurality of heat heatmaps highlights those regions which are most critical for a prediction of whether the tissue contains a lesion or not.

Example 9: The method of one of the preceding examples, wherein at least one of the first and second models is a machine learning model, and/or the first model is or comprises a convolutional neural network, and/or the second model comprises a transformer-based model and optionally a convolutional neural network for preprocessing the input of the transformer-based model.

Example 10: The method of one of the preceding examples 4 to 9, wherein at least one of the first, second, superimposed, and the plurality of heat heatmaps is generated using the transformer-based model and/or the attention-based model.

Example 11: The method of one of the preceding examples 9 and 10, wherein the convolutional neural network of the first model is trained to identify a particular tissue type in a plurality of training data sets of digital histological images of human or animal tissue, wherein
training of the convolutional neural network comprises performing with the plurality of training data sets the steps of:
selecting a target tissue area of a training data set,
dividing the target tissue area into a first set of tiles of constant size and having a first image magnification,
dividing the target tissue area into at least a second set of tiles of constant size and having a second image magnification different from the first image magnification,
inputting the at least two sets of tiles into the convolutional neural network, wherein the convolutional neural network is an at least two-headed convolutional neural network in which the at least two sets of tiles are processed in parallel whereby the features of the at least two sets of tiles are concatenated, and
labelling the output results of the convolutional neural network with respect to the target tissue type.

Example 12: The method of one of the preceding examples, wherein the identified different tissue types are tissues of different organs.

Example 13: The method of one of the preceding examples, wherein dividing the target tissue area into the at least two tile sets comprises: extracting a foreground mask of the tissue region, providing annotations classifying areas of the tissue region, and merging the annotations with the foreground mask.

Example 14: A computer program comprising computer-readable instructions which when executed by a data processing system cause the data processing system to carry out the method according to any one of preceding method examples.

Example 15: A system for predicting and/or detecting the presence of a lesion in a human or animal tissue, the system comprising a processing unit configured to: input image data of human or animal tissue into a first model, wherein in response the first model identifies a tissue type, and predict the presence of a lesion based on the image data and the tissue type using a second model.

1. A computer-implemented method of predicting and/or detecting presence of a lesion in human or animal tissue, the method comprising:
identifying a tissue type based on image data of the human or animal tissue using a first model; and
predicting and/or detecting the presence of the lesion in the tissue based on the image data and the tissue type using a second model.

2. The method of claim 1, wherein:
identifying the tissue type comprises inputting the image data into the first model, wherein in response the first model identifies the tissue type; and/or
predicting and/or detecting the presence of the lesion comprises inputting the image data into the second model, the image data being annotated with the tissue type and/or comprise data representing the tissue type, wherein in response the second model predicts the presence of the lesion in the tissue.

3. The method of claim 1, wherein:
predicting and/or detecting the presence of the lesion comprises predicting and/or detecting at least one of: a probability of the presence of the lesion, a lesion type, and/or a degree of lesion severity.

4. The method of claim 1, wherein:
predicting and/or detecting the presence of the lesion comprises predicting and/or detecting for each of a plurality of different tissue regions a probability of the presence of the lesion; and/or
predicting and/or detecting the presence of the lesion comprises generating a first heatmap of the tissue, wherein the first heatmap indicates for each of the plurality of different tissue regions the probability of the presence of the lesion.

5. The method of claim 4, wherein:
predicting and/or detecting the presence of the lesion comprises generating a second heatmap of the tissue, wherein the second heatmap indicates for each of the plurality of different tissue regions a probability of the presence of no lesion.

6. The method of claim 5, wherein:
predicting and/or detecting the presence of the lesion further comprises generating a superimposed heatmap of the tissue combining the first heatmap with the second heatmap by superimposition.

7. The method of claim 6, wherein:
at least one of the first, second, and superimposed heatmaps is related to the probability of the presence of a particular lesion type; and/or
predicting and/or detecting the presence of the lesion further comprises generating a plurality of heatmaps, wherein each heatmap is related to the probability of the presence of the particular lesion type.

8. The method of claim 7, wherein:
at least one of the first, second, superimposed, and the plurality of heatmaps highlights those regions which are most critical for a prediction of whether the tissue contains the lesion or not.

9. The method of claim 1, wherein:
at least one of the first and second models is a machine learning model; and/or
the first model is or comprises a convolutional neural network; and/or
the second model comprises a transformer-based model and optionally a convolutional neural network for preprocessing input of the transformer-based model.

10. The method of claim 7, wherein:
at least one of the first, second, superimposed, and the plurality of heatmaps is generated using a transformer-based model and/or an attention-based model.

11. The method of claim 9, wherein:
the convolutional neural network of the first model is trained to identify a particular tissue type in a plurality of training data sets of digital histological images of human or animal tissue, wherein:
training of the convolutional neural network comprises performing with the plurality of training data sets:
selecting a target tissue area of a training data set;
dividing the target tissue area into a first set of tiles of constant size and having a first image magnification;
dividing the target tissue area into at least a second set of tiles of constant size and having a second image magnification different from the first image magnification;
inputting the at least first and second sets of tiles into the convolutional neural network, wherein the convolutional neural network is an at least two-headed convolutional neural network in which the at least first and second sets of tiles are processed in parallel wherein features of the at least first and second sets of tiles are concatenated; and
labelling output results of the convolutional neural network with respect to a target tissue type.

12. The method of claim 11, wherein:
identified different tissue types are tissues of different organs.

13. The method of claim 11, wherein:
dividing the target tissue area into the at least first and second sets of tiles comprises:
extracting a foreground mask of a tissue region;
providing annotations classifying areas of the tissue region; and
merging the annotations with the foreground mask.

14. A computer program comprising computer-readable instructions, which when executed by a data processing system, cause the data processing system to carry out the method of claim 1.

15. A system for predicting and/or detecting presence of a lesion in human or animal tissue, the system comprising a processing unit configured to:
input image data of the human or animal tissue into a first model, wherein in response the first model identifies a tissue type; and
predict the presence of the lesion based on the image data and the tissue type using a second model.
